(19) United States
(12) Patent Application Publication
(10) Pub. No.: US 2017/0368542 A1
(43) Pub. Date: Dec. 28, 2017

(54) SILICOALUMINOPHOSPHATE CATALYST FOR CHLOROMETHANE CONVERSION

(71) Applicant: SABIC Global Technologies B.V., Amsterdam (NL)

(72) Inventors: Ashim Kumar Ghosh, Sugar Land, TX (US); Alla Khanmamedova, Sugar Land, TX (US); Mike Mier, Sugar Land, TX (US); Jonathan Banke, Sugar Land, TX (US)

(73) Assignee: SABIC Global Technologies B.V., Amsterdam (NL)

(21) Appl. No.: 15/533,608

(22) PCT Filed: Nov. 18, 2015
(86) PCT No.: PCT/US2015/061344

§ 371 Date: Jun. 6, 2017

Related U.S. Application Data
(60) Provisional application No. 62/093,205, filed on Dec. 17, 2014.

(51) Int. Cl. B01J 29/85; C07C 11/06; B01J 35/00; B01J 29/90; C01B 39/54; C01B 37/08; C07C 1/26; C07C 11/04

(52) U.S. Cl. CPC B01J 29/85; C07C 1/26; C07C 11/06; C07C 11/04; B01J 29/90; C01B 39/54; C01B 37/08; B01J 35/002; C07C 2529/85

(57) ABSTRACT

Disclosed is a catalyst capable of producing an olefin from an alkyl halide, the catalyst comprising a silicoaluminophosphate (SAPO) having a chabazite zeolite structure with the following chemical composition (SiₓAl_{y}P_{z})O₂, where x, y, and z represent the mole fractions of silicon, aluminum, and phosphorus, respectively, present as tetrahedral oxides, x is 0.01 to 0.30 and the sum of x+y+z is 1, and where the catalyst comprises silicon tetrahedral oxides that are connected with three or less aluminum tetrahedral oxide as shown by ²⁹Si magic angle spinning (MAS) nuclear magnetic resonance (NMR) spectroscopy peak(s) with peak(s) maxima between −93 ppm and −115 ppm.

### CROSS REFERENCE TO RELATED APPLICATIONS

This application claims the benefit of priority of U.S. Provisional Patent Application No. 62/093,205, filed Dec. 17, 2014, which is hereby incorporated by reference in its entirety.

### BACKGROUND OF THE INVENTION

### A. Field of the Invention

The invention generally concerns the use of small pore size silicoaluminophosphate (SAPO) catalysts to produce light olefins from alkyl halides. In particular, the SAPO catalysts have a chabazite zeolite structure containing SiO₄ tetrahedra connected to three or less AlO₄ tetrahedra and exhibit improved stability and catalytic performance over prolong periods of use.

### B. Description of Related Art

Descriptions of units, abbreviation, terminology, etc. used throughout the present invention are summarized in Table 1.

Light olefins such as ethylene and propylene are used by the petrochemical industry to produce a variety of key chemicals that are then used to make numerous downstream products. By way of example, both of these olefins are used to make a multitude of plastic products that are incorporated into many articles and goods of manufacture. FIGS. 1A and 1B provide examples of products generated from ethylene (FIG. 1A) and propylene (FIG. 1B).

Methane activation to higher hydrocarbons, especially to light olefins, has been the subject of great interest over many decades. Recently, the conversion of methane to light olefins via a two-step process that includes conversion of methane to methyl halide, particularly to methyl mono-halide, for example, to methyl chloride followed by conversion of the halide to light olefins has attracted great attention. Zeolite (e.g., ZSM-5) or zeolite type catalysts (e.g., SAPO-34) have been tried for methyl chloride (or other methyl halide) conversion. However, the selectivity to a desired olefin (e.g., propylene) and the rapid catalyst deactivation for the halide reaction remain the major challenges for commercial success.

One of the most commonly used catalysts in the petrochemical industry is ZSM-5 zeolite. It is a medium pore zeolite with pore size about 5.5 Å and is shown to convert methyl halide, particularly methyl chloride or methyl bromide, to C₂-C₄ olefins and aromatics under methyl halide reaction conditions. Whereas, molecular sieve SAPO-34, an isostructure of chabazite zeolite, having small pore opening (3.8 Å) is shown to convert methyl halide to ethylene and propylene and small amounts of C₄ olefins. However, both catalysts are shown to deactivate rapidly during methyl halide conversion due to carbon deposition on the catalysts.

The production of SAPO-34 catalysts can be complicated and can depend on the silicon source, structuring directing agent, crystallization condition, and material composition in initial gel formation, which can influence the average crystal size of the catalyst. (See, for example, Askari et al. in “Reviews on Advancement of Material Science, 2012, Vol. 32, pp. 83-93). U.S. Patent Application Publication No. 2012/0203046 to Chae et al. attempts to solve the problems of commercial catalyst for production of olefins from oxygenated compounds by development of a microsphere SAPO catalyst. The preparation of this catalyst, however, uses multiple structure directing agents and produces a higher amount of ethylene than propylene from oxygen containing starting materials. U.S. Pat. No. 6,403,855 to Mertens attempts to prepare a zeolite catalyst for the production of olefins from oxygenated feed stocks by seeding a precursor gel with chabazite crystals with different forms of stirring. This patent, however, does not provide any data on the use of the catalyst in the production of olefins. U.S. Pat. No. 6,953,767 to Janssen et al. describes a SAPO-34 catalyst suitable for production of olefins from oxygen containing starting materials with selectivity to C₄ olefins being greater than 20%. Tan et al. in Microporous and Mesoporous Materials, 2002, Vol. 53, pp. 97-108 describes the mechanism of Si incorporation in SAPO-34 structure during hydrothermal synthesis of SAPO-34 molecular sieves. As-synthesized SAPO-34 (collected between 1.0 to 2.0 h) showed ²⁹Si MAS NMR a strong peak at −91 ppm attributing to the incorporation of Si in SAPO-34 structure as Si(4Al) (i.e., Si atom is bonded with four Al atoms via oxygen atoms). At a longer crystallization time (2.5 h or longer) the SAPO-34 shows additional four ²⁹Si MAS NMR weak peaks at −94, −100, −104 and −110 ppm attributing to the presence of Si atoms bonded with 3, 2, 1 and 0 aluminum (Al) atoms, respectively, via oxygen atoms. This paper, however, does not provide any data or suggestion on the use of the catalyst in the production of olefins.

While the currently available SAPO-34 catalysts have good selectivity for both ethylene and propylene from oxygenated feed stocks, a major problem with these SAPO-34 catalysts is their lack of stable catalytic performance over prolonged periods of use for the alkyl halide conversion. Notably, the currently available SAPO-34 catalysts show methyl chloride conversion rates of less than 20% after being used for 20 h. Such deactivation of these catalysts require frequent or continuous catalyst regeneration, or frequent catalyst change-out resulting in inefficient plant operation, or use of more catalysts to produce the desired amounts of ethylene and propylene, which in turn increases the manufacturing costs. Still further, the catalytic material has to be re-supplied in shorter time intervals, which oftentimes requires the reaction process to be shut down. This also adds to the inefficiencies of the currently available SAPO-34 catalysts.

TABLE 1

| Abbreviation | Description |
|---|---|
| Å | Angstrom |
| Avg. | Average |
| BET SA | BET (Brunauer-Emmett-Teller) surface area |
| ° C. | degree Celsius |
| ° C./min | degree Celsius per minute |
| cm³/min | cubic centimeter per min |
| g | gram |
| g/cm³ | gram per cubic centimeter |
| h | hour |
| m²/g | meter square per gram |
| mol % | mole percent |
| mmol/g-cat | millimole per gram of catalyst |
| % | percent |
| psig | pound per square inch gauge |
| SAR | silica to alumina ratio |
| XRF | X-ray fluorescence |
| WHSV | weight hourly space velocity |
| wt. % | weight percent |

### SUMMARY OF THE INVENTION

A discovery has been made that solves the rapid catalyst deactivation problems associated with small pore size molecular sieve catalysts (e.g., silicoaluminophosphate (SAPO-34)) without compromising their selectivity for C₂-C₄ olefin production. This discovery is premised on a SAPO catalyst containing SiO₄ tetrahedra connected with three or less AlO₄ tetrahedra, and having improved stability when converting alkyl halides (e.g., methyl halide) to light olefins (e.g., ethylene and propylene). This enhanced stability allows for a more efficient process of making light olefins when compared with the currently available SAPO catalysts. For instance, less catalytic material is needed and the frequency in regenerating spent catalysts is reduced without compromising the selectivity of light olefin production.

In one aspect of the present invention, there is disclosed a catalyst capable of producing an olefin form an alkyl halide, the catalyst can include a SAPO framework structure containing a small pore opening. In particular the SAPO framework structure can be chabazite zeolite structure containing silicon tetrahedra (SiO₄) connected with three or less aluminum tetrahedral (AlO4). The catalyst exhibits ²⁹Si peaks with peak(s) maxima between −93 ppm and −115 ppm when analyzed using ²⁹Si magic angle spinning (MAS) nuclear magnetic resonance (NMR) spectroscopy. In a particular aspect of the invention, silicon atom is connected to 3 or less aluminum atoms (e.g., Si(nAl) structures, where n=0 to 3) by sharing oxygen atoms. In particular instances, the catalyst exhibits ²⁹Si peaks with peak(s) maxima −93 ppm and −97 pm, which indicates n=3. In certain instances, the majority of the silicon tetrahedra (SiO₄) units are connected with three aluminum tetrahedral (AlO4). The catalyst of the present invention can have the chemical composition or formula (I) shown below.

(SiₓAl_{y}P_{z})O₂ (I)

where x, y, and z represent the mole fractions of silicon, aluminum, and phosphorus, respectively, present as tetrahedral oxides, and x is 0.01 to 0.3 and the sum of x+y+z is 1, and where each silicon tetrahedral oxide is connected to 3 or less aluminum tetrahedral oxides, as shown by ²⁹Si MAS NMR peak(s) with peak(s) maxima between −93 ppm and −115 ppm. The catalyst can have a mole fraction of aluminum (y) of 0.4 to 0.6 and a mole fraction of phosphorus (z) is 0.25 to 0.49. In a particular instance, the catalyst has an elemental Si content of 3.75 to 4.25 wt. %, an elemental Al content of 24.50 to 27.50 wt. % and an elemental phosphorus content of 15.0 to 17.5 wt. %. In a particular aspect of the invention, a majority of the silicon tetrahedra (SiO₄) in the SAPO framework are connected with three or less aluminum tetrahedra (AlO₄). Such a catalyst can include 25% or less, 15% or less, or 10% or less of SiO₄ tetrahedra connected by four AlO₄ tetrahedra. In yet another embodiment, the catalyst is characterized as having a powder x-ray diffraction pattern as substantially depicted in Table 6 of this application or Table 7 of this application.

In certain aspects, the SAPO catalysts are capable of converting 30 to 95% of the alkyl halide after 20 hours of use at a temperature of 325 to 375° C., or about 350° C., a WHSV of the halide feed of greater than 0.5 h⁻¹ or of between 0.5 and 6.0 h⁻¹, and a pressure of 1 to 4 psig. The catalysts can have a selectivity of C₂-C₃ olefins of at least 80% after 20 hours of use at a temperature of 300° C. to 375° C. In certain aspects, the selectivity of ethylene can be at least 40%, and the selectivity of propylene can be at least 45% after 20 hours of use at a temperature of 300° C. to 375° C. In preferred aspects, the combined selectivity of ethylene and propylene can be at least 80%, or at least 90% at 10% alkyl halide conversion at 300° C. to 375° C.

The decrease of alkyl halide conversion can be attributed to carbon deposition on the SAPO catalyst. The carbon deposition causes the blockage of active sites resulting in decrease of conversion. The spent catalyst can be regenerated by burning of the deposited carbon. Such carbon burning can generally be performed by heating the spent catalyst under oxygen, preferably diluted oxygen, often used air diluted with nitrogen, at temperature between 400° C. and 600° C.

In still another embodiment of the present invention there is disclosed a system for producing olefins. The system can include an inlet for a feed comprising the alkyl halide discussed above and throughout this specification, a reaction zone that is configured to be in fluid communication with the inlet, wherein the reaction zone comprises any one of the SAPO catalysts of the present invention that are discussed above and throughout this specification, and an outlet configured to be in fluid communication with the reaction zone to remove an olefin hydrocarbon product from the reaction zone. During use, the reaction zone can further include the alkyl halide feed and the olefin hydrocarbon product (e.g., ethylene, propylene, and/or butylene). The temperature of the reaction zone can be 325 to 375° C. The system can include a collection device that is capable of collecting the olefin hydrocarbon product.

Also disclosed is a method of synthesizing any one of the silicoaluminophosphate (SAPO) catalysts of the present invention. The method can include making a gel containing the sources of Si, Al, and P, and a structure directing agent, and heating the gel mixture at a temperature of 200° C. to 225° C. under conditions to produce the SAPO catalyst. In some embodiments, the gel mixture is heated under static conditions for about 24 hours. In other embodiments, the gel mixture is heated with agitation for about 24 hours. A non-limiting example of a silica containing material includes colloidal silica. A non-limiting example of an aluminum containing material includes aluminum isopropoxide. A non-limiting example of a phosphorus containing material includes phosphoric acid. A non-limiting example of a structure directing agent includes tetraethylammonium hydroxide, which can be added to the mixture prior to heating the mixture. The synthesized SAPO catalyst can be further separated from the mixture and washed with water followed by drying around 100° C. The process can further include calcining the produced SAPO catalyst at a temperature of 400 to 600° C. for more than 0.5 h, preferably more than 2 h and less than 20 h.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The catalysts of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the catalysts of the present invention are their ability to selectivity produce light olefins, and in particular, ethylene and propylene, in high amounts, while also remaining stable/activated after prolonged periods of use (e.g., 20 hours).

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIGS. 1A and 1B depict illustrations of various chemicals and products that can be produced from ethylene (FIG. 1A) and propylene (FIG. 1B).

FIG. 2 depicts a system for producing olefins from alkyl halides.

FIG. 3 depicts ²⁹Si MAS NMR spectrum of the comparative catalysts A and B. (Peak intensities are in arbitrary units and “not to scale.”)

FIG. 4 depicts ²⁹Si MAS NMR spectra of the catalysts C and D of the present invention. (Peak intensities are in arbitrary units and “not to scale.”)

FIG. 5 depicts ²⁷Al MAS NMR spectra of catalysts A-D. (Peak intensities are in arbitrary units and “not to scale.”)

FIG. 6 depicts ³¹P MAS NMR spectra of catalysts A and D. (Peak intensities are in arbitrary units and “not to scale.”)

FIG. 7 is a graph of time on stream in hours versus percentage of methyl chloride conversion.

### DETAILED DESCRIPTION OF THE INVENTION

The currently available SAPO catalysts, particularly SAPO-34 catalysts, show high activity for alkyl halide conversion with selectivity to light olefins (e.g., ethylene and propylene). These types of catalysts, however, tend to deactivate rapidly during the initial periods of the reaction reaching an unacceptable level of alkyl halide conversion within hours. This rapid deactivation leads to a number of processing and cost inefficiencies.

A discovery has been made, which results in SAPO catalysts having improved stability and high selectivity for C₂-C₄ olefins. The stability and selectivity is obtained by using SAPO catalysts that contain Si atoms bonded with 3 or less aluminum atoms via oxygen atoms in a silicon tetra-oxide molecule. This improved stability results in a more efficient and continuous productions of light olefins from alkyl halides without having to continuously regenerate spent catalysts or constantly provide additional catalysts to the reaction process. Further, the catalysts have been shown to have at least 80% selectivity of C₂-C₄ olefins.

These and other non-limiting aspects of the present invention are discussed in further detail in the following sections.

### A. SAPO Catalysts

The SAPO catalysts have an open microporous structure with regularly sized channels, pores or “cages.” These materials are sometimes referred to as “molecular sieves” in that they have the ability to sort molecules or ions based primarily on the size of the molecules or ions. SAPO materials are both microporous and crystalline and have a three-dimensional crystal framework of PO₄+, AlO₄ and SiO₄ tetrahedra. The SAPO catalysts of the present invention are designed such that they have an chabazite zeolite structure containing SiO4 tetrahedra each connected with 3 or less AlO4 tetrahedra. The chemical formula of the SAPO catalysts of the present invention is given in formula (I) above.

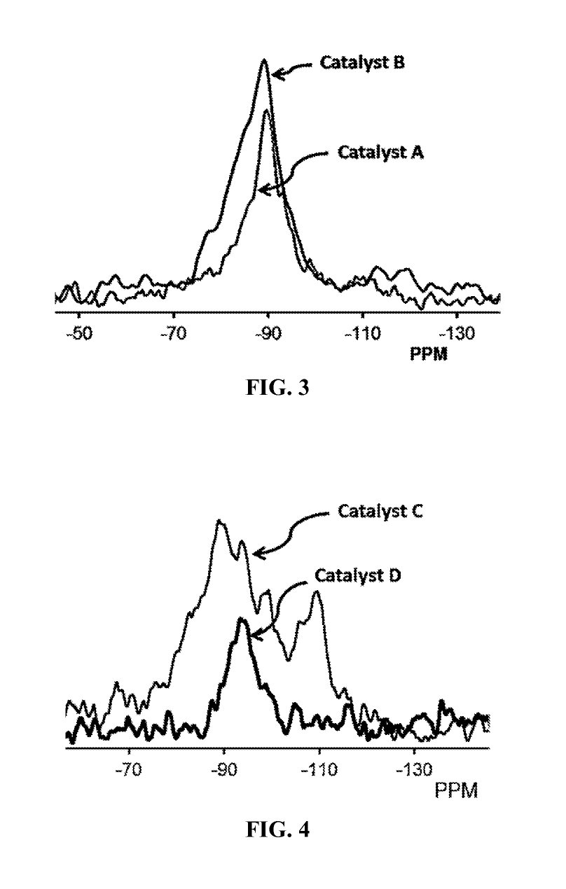

SAPO catalyst are made by using a gel containing aluminum (Al), phosphorus (P) and silicon (Si) compounds in the presence of a structure directing agent under crystallization conditions. In the SAPO framework, the Si, Al, and P are bonded to each other by sharing oxygen atoms forming tetrahedra TO₄ (T=Si, Al, P). It was discovered in the context of the present invention that certain processing conditions discussed below and in the Examples resulted in SAPO-34 catalysts that contained SiO₄ tetrahedra in the framework structure each being connected with three or less AlO₄ units (see FIG. 4). It was further discovered that such a crystal lattice structure increases the stability of the catalyst when compared with typical SAPO-34 catalysts having SiO₄ units surrounded by four AlO₄ units. The following illustrates various possible SiO₄ environments in the framework of the SAPO-34 catalysts of the present invention (note that an oxygen atom is shared between each Al—P, Al—Si, and Si—Si bonds; boxes are used to highlight Si atoms; superscripts indicate the number of AlO₄ units surrounding any given SiO₄ unit):

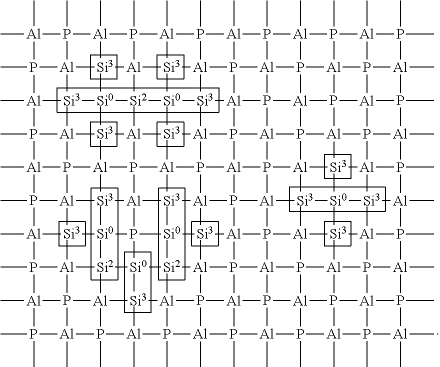

Without wishing to be bound by theory, it is believed that the formation of Si(nAl) structures, where n=0 to 3, the intrinsic acidity of SAPO catalyst will increase due to the presence of SiO₄ tetrahedra connected with 3 or less AlO₄ tetrahedra which increases the charge unbalance at the framework structure. The increase of intrinsic acidity of the SAPO catalysts can increase the catalytic activity and stability of the catalyst in the formation of light olefins from alkyl halides.

Non-limiting examples of making SAPO catalysts of the present invention are provided in the Examples section. A general non-limiting method of making the SAPO catalysts includes preparing an aqueous mixture of aluminum iso-propoxide with phosphoric acid and, optionally hydrochloric acid. Colloidal silica can be added to the aluminum/phosphorous mixture with agitation followed by the addition of tetraethylammonium hydroxide. The gel mixture can be aged overnight at room temperature with or without agitation. The aged gel can then be heated at 200 to 215° C. for a desired amount of time in an autoclave with or without agitation. The formed SAPO material can be separated and washed with water, and dried at about 90° C. The dried material can be sieved through an appropriate mesh screen (for example, 40 mesh) and calcined in air at 500-600° C.

### B. Alkyl Halide Feed

The alkyl halide feed includes one or more alkyl halides. The alkyl halide feed may contain alkyl mono halides, alkyl dihalides, alkyl trihalides, preferably alkyl mono halide with less than 10% of other halides relative to the total halides. The alkyl halide feed may also contain nitrogen, helium, steam, and so on as inert compounds. The alkyl halide in the feed may have the following structure: CₙH₍₂ₙ₊₂₎₋ₘXₘ, where n and m are integers, n ranges from 1 to 5, preferably 1 to 3, even more preferably 1, m ranges 1 to 3, preferably 1, X is Br, F, I, or Cl. Non-limiting examples of alkyl halides include methyl chloride, methyl bromide, methyl fluoride, or methyl iodide, or any combination thereof. In particular aspects, the feed may include about 10, 15, 20, 40, 50 mole % or more of the alkyl halide. In particular embodiments, the feed contains up to 20 mole % of the feed includes an alkyl halide. In preferred aspects, the alkyl halide is methyl chloride. In a particular embodiment, the alkyl halide is methyl chloride or methyl bromide.

The production of alkyl halide, particularly of methyl chloride (CH₃Cl, See Equation (V) below), is commercially produced by thermal chlorination of methane at 400° C. to 450° C. and at a raised pressure. Catalytic oxychlorination of methane to methyl chloride is also known. In addition, methyl chloride is industrially made by reaction of methanol and HCl at 180° C. to 200° C. using a catalyst. Alternatively, methyl halides are commercially available from a wide range of sources (e.g., Praxair, Danbury, Conn.; Sigma-Aldrich Co. LLC, St. Louis, Mo.; BOC Sciences USA, Shirley, N.Y.). In preferred aspects, methyl chloride and methyl bromide can be used alone or in combination.

### C. Olefin Production

The SAPO catalysts of the present invention help to catalyze the conversion of alkyl halides to C₂-C₄ olefins such as ethylene, propylene and butenes. The following non-limiting two-step process is an example of conversion of methane to methyl chloride and conversion of methyl chloride to ethylene, propylene and butylene. The second step illustrates the reactions that are believed to occur in the context of the present invention:

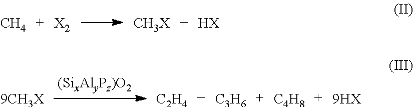

where X is Br, F, I, or Cl, and where x, y, and z represent the mole fractions of silicon, aluminum, and phosphorus, respectively, present as tetrahedral oxides, x is 0.01 to 0.3 and the sum of x+y+z is 1, and where each silicon tetrahedral oxide is covalently connected to 3 or less aluminum tetrahedral oxides. Besides the C₂-C₄ olefins the reaction may produce byproducts such as methane, C₅ olefins, C₂-C₅ alkanes and aromatic compounds such as benzene, toluene and xylene.

Conditions sufficient for olefin production (e.g., ethylene, propylene and butylene as shown in Equation (III)) include temperature, time, alkyl halide concentration, space velocity, and pressure. The temperature range for olefin production may range from about 300° C. to 500° C., preferably ranging 350° C. to 450° C. A weight hourly space velocity (WHSV) of alkyl halide higher than 0.5 h⁻¹ can be used, preferably between 1.0 and 6.04 h⁻¹, more preferably between 2.0 and 3.5 h⁻¹. The conversion of alkyl halide is carried out at a pressure less than 5 psig preferably less than 1 psig, more preferably less than 0.5 psig, or at atmospheric pressure. The conditions for olefin production may be varied based on the type of the reactor.

The reaction can be carried out over the catalyst of the present invention for prolonged periods of time without changing or re-supplying new catalyst or catalyst regeneration. This is due to the stability or slower deactivation of the catalysts of the present invention. Therefore, the reaction can be performed for a period until the level of alkyl halide conversion reaches to a preset level (e.g., 20%). In preferred aspects, the reaction is continuously run for 20 h or 20 h to 50 h or longer without having to stop the reaction to resupply new catalyst or catalyst regeneration. The method can further include collecting or storing the produced olefin hydrocarbon product along with using the produced olefin hydrocarbon product to produce a petrochemical or a polymer.

### D. Catalyst Activity/Selectivity

Catalytic activity as measured by alkyl halide conversion can be expressed as the % moles of the alkyl halide converted with respect to the moles of alkyl halide fed. In particular aspects, the combined selectivity of ethylene, propylene and butylene is at least 50% under certain reaction conditions. In certain instances, the selectivity of propylene is about 30% or higher, the selectivity of butylene is about 10% or higher, and ethylene selectivity is about 12% or less. As an example, methyl chloride (CH₃Cl) is used here to define conversion and selectivity of products by the following equations (VII)-(X):

$$\% {CH}_{3} Cl Conversion = \frac{( {CH}_{3} Cl ) ° - ( {CH}_{3} Cl )}{( {CH}_{3} Cl ) °} \times 100$$

where, (CH₃Cl)° and (CH₃Cl) are moles of methyl chloride in the feed and reaction product, respectively.

Selectivity is defined as C-mole % and are defined for ethylene, propylene, and so on as follows):

$$\% Ethylene Selectivity = \frac{2 ( {C}_{2} {H}_{4} )}{\begin{matrix}( {CH}_{4} ) + 2 ( {C}_{2} {H}_{4} ) + 2 ( {C}_{2} {H}_{6} ) + \\ 3 ( {C}_{3} {H}_{6} ) + 3 ( {C}_{3} {H}_{8} ) + 4 ( {C}_{4} {H}_{8} ) + 4 ( {C}_{4} {H}_{10} ) +\end{matrix}} \times 100 \qquad ( V )$$

where, the numerator is the carbon adjusted mole of ethylene and the denominator is the sum of all the carbon adjusted mole of all hydrocarbons in the product stream.

Selectivity for propylene may be expressed as:

$$\% Propylene Selectivity = \frac{3 ( {C}_{3} {H}_{6} )}{\begin{matrix}( {CH}_{4} ) + 2 ( {C}_{2} {H}_{4} ) + 2 ( {C}_{2} {H}_{6} ) + \\ 3 ( {C}_{3} {H}_{6} ) + 3 ( {C}_{3} {H}_{8} ) + 4 ( {C}_{4} {H}_{8} ) + 4 ( {C}_{4} {H}_{10} ) +\end{matrix}} \times 100 \qquad ( VI )$$

where, the numerator is the carbon adjusted mole of propylene and the denominator is the sum of all the carbon adjusted mole of all hydrocarbons in the product stream.

Selectivity for butylene may be expressed as:

$$\% Butylene Selectivity = \frac{4 ( {C}_{4} {H}_{8} )}{\begin{matrix}( {CH}_{4} ) + 2 ( {C}_{2} {H}_{4} ) + 2 ( {C}_{2} {H}_{6} ) + \\ 3 ( {C}_{3} {H}_{6} ) + 3 ( {C}_{3} {H}_{8} ) + 4 ( {C}_{4} {H}_{8} ) + 4 ( {C}_{4} {H}_{10} ) +\end{matrix}} \times 100 \qquad ( VII )$$

where, the numerator is the carbon adjusted mole of butylene and the denominator is the sum of all the carbon adjusted mole of all hydrocarbons in the product stream.

### E. Olefin Production System

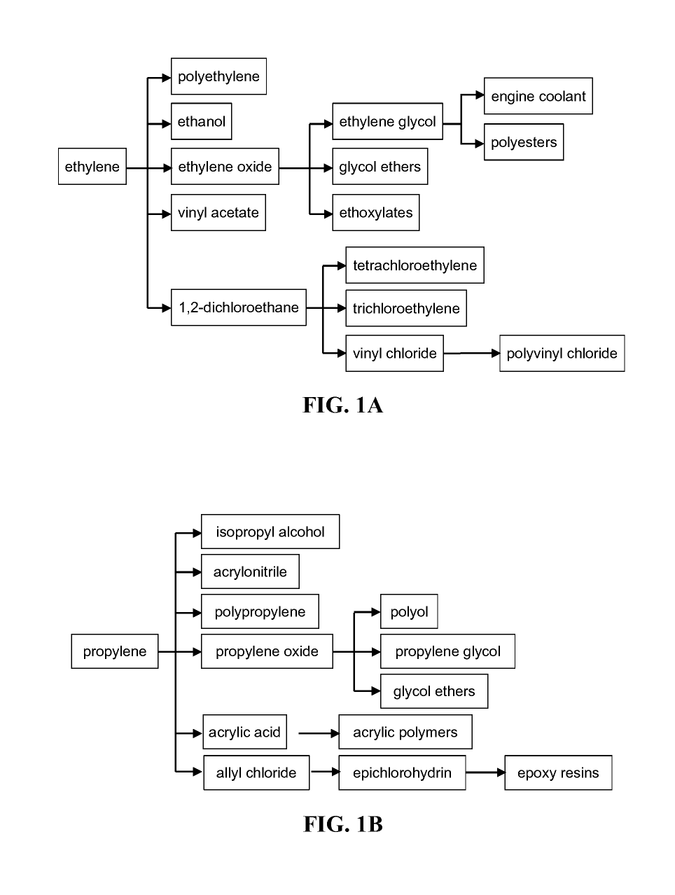

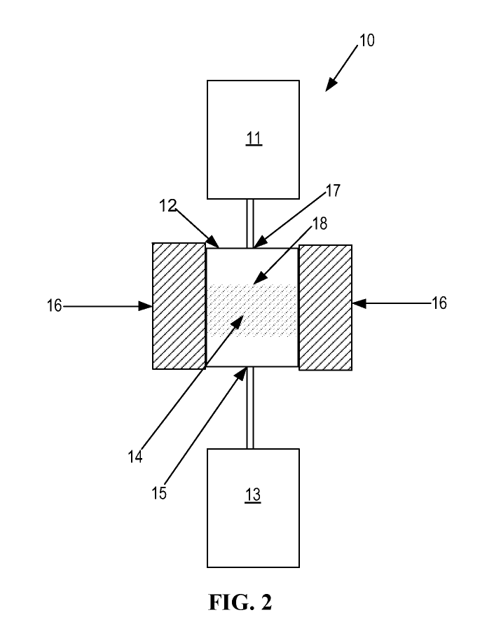

Referring to FIG. 2, a system 10 is illustrated, which can be used to convert alkyl halides to olefin hydrocarbon products with the SAPO zeolite catalysts of the present invention. The system 10 can include an alkyl halide source 11, a reactor 12, and a collection device 13. The alkyl halide source 11 can be configured to be in fluid communication with the reactor 12 via an inlet 17 on the reactor. As explained above, the alkyl halide source can be configured such that it regulates the amount of alkyl halide feed entering the reactor 12. The reactor 12 can include a reaction zone 18 having the SAPO zeolite catalyst 14 of the present invention. The amounts of the alkyl halide feed 11 and the catalyst 14 used can be modified as desired to achieve a given amount of product produced by the system 10. Non-limiting examples of reactors that can be used include fixed-bed reactors, fluidized bed reactors, bubbling bed reactors, slurry reactors, rotating kiln reactors, or any combinations thereof when two or more reactors are used. In preferred aspects, reactor 12 that can be used is a fixed-bed reactor (e.g., a fixed-bed tubular quartz reactor which can be operated at atmospheric pressure). The reactor 12 can include an outlet 15 for products produced in the reaction zone 18. The products produced can include ethylene, propylene and butylene. The collection device 13 can be in fluid communication with the reactor 12 via the outlet 15. Both the inlet 17 and the outlet 15 can be open and closed as desired. The collection device 13 can be configured to store, further process, or transfer desired reaction products (e.g., C₂-C₄ olefins) for other uses. By way of example only, FIG. 1 provides non-limiting uses of propylene produced from the catalysts and processes of the present invention. Still further, the system 10 can also include a heating source 16. The heating source 16 can be configured to heat the reaction zone 18 to a temperature sufficient (e.g., 325 to 375° C.) to convert the alkyl halides in the alkyl halide feed to olefin hydrocarbon products. A non-limiting example of a heating source 16 can be a temperature controlled furnace. Additionally, any unreacted alkyl halide can be recycled and included in the alkyl halide feed to further maximize the overall conversion of alkyl halide to olefin products. Further, certain products or byproducts such as butylene, C₅₊ olefins and C₂₊ alkanes can be separated and used in other processes to produce commercially valuable chemicals (e.g., propylene). This increases the efficiency and commercial value of the alkyl halide conversion process of the present invention.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results. The materials used in the following examples are described in Table 2, and were used as-described unless specifically stated otherwise.

TABLE 2

| Material | Source |
|---|---|
| SAPO-34 (powder form)^{a} | ACS Material |
| Colloidal Silica (Ludox SM-30), 30 wt. % SiO₂ | Sigma-Aldrich ® |
| Aluminum iso-propoxide (Al(O - i-Pr)₃), ≧98% purity | Sigma-Aldrich ® |
| Phosphoric acid (H₃PO₄ (85 wt. % in aqueous)) | Sigma Aldrich ® |
| Hydrochloric acid (HCl), 37 wt. % HCl in aqueous | Sigma-Aldrich ® |
| Tetraethylammonium hydroxide ((C₂H₅)₄N(OH)), | Sigma Aldrich ® |
| 35 wt. % in aqueous | |
| Water (deionized) | SABIC labs |

^{a}SAPO-34 obtained from ACS Material, Medford, MA, USA.

### Examples 1-4

### Preparation of Comparative Catalysts A and B

Catalyst A:

A SAPO-34 powder was obtained from a commercial source (ACS Material). The SAPO-34 powder was calcined in air at 550° C. for 2 h and designated as Catalyst A.

Catalyst B:

was made by combining 27.2 g of aluminum isopropoxide with 36.36 g of water, 13.72 g of phosphoric acid and 2.30 g of hydrochloric acid with vigorous stirring for about 35 min. To this mixture 4.0 g colloidal silica (Ludox SM-30) was added and then 56.24 g tetraethyl ammonium hydroxide was added. The mixture was aged at elevated temperature 80° C. with stirring in a Teflon lined autoclave for a prolonged time (5 days). Crystallization was done in Teflon lined 300 ml static autoclave at 215° C. for 24 h. Formed SAPO material was separated, washed with water, and dried at 90° C. overnight. Material was then sieved through a 40 mesh screen and calcined at 600° C. in air for 3 hours. This was designated as Catalyst B.

### Preparation of Catalysts of the Present Invention C and D

Catalyst C:

A mixture was prepared by adding 13.65 g aluminum isopropoxide to 18.15 g water in a Teflon liner, stirred at 60° C. in a hot water bath for 2 h. The isopropoxide mixture was cooled to room temperature and a mixture of phosphoric acid and hydrochloric acid (5.49 g H3PO4 and 1.55 g HCl) was added drop wise while stirring. Colloidal silica (4.00 g Ludox SM-30) was added to the mixture while stirring and 28.12 g tetraethylammonium hydroxide was slowly added while vigorously stirring. The gel mixture was aged overnight at room temperature without stirring. The gel was heated at 215° C. for 99 h without stirring. The formed SAPO material was separated and washed with water, and dried at 90° C. overnight. The material was sieved through 40 mesh screen and calcined in air at 600° C. for 2 h. This was designated as catalyst C.

Catalyst D:

A mixture was prepared by adding 27.75 g of aluminum isopropoxide to solution of 13.80 g of phosphoric acid in 41 g of deionized water with following stirring for about 1.5 hour. Colloidal silica (4.00 g Ludox SM-30) was then added with stirring for 15 minutes. Then 33.6 g of 35% tetraethylammonium hydroxide were added and the mixture was stirred at room temperature for 19 hours. Crystallization was made in a stirred Teflon lined Parr autoclave at 200° C. for 24 hours. Formed SAPO material was separated, washed with water, and dried at 90° C. overnight. Material was then sieved through a 40 mesh screen and calcined at 600° C. in air for 2 hours. This was designated as catalyst D.

### Analysis of Catalysts A-D

All catalysts were analyzed for Si, Al and P by XRF techniques and the results are shown in Table 3. The BET surface area (BET SA) and micropore area were measured by BET N₂ adsorption at −196° C. The results are shown in Table 3.

TABLE 3
Elemental analysis and N₂ adsorption data

| | | | | | | | N₂ Adsorption | |
|---|---|---|---|---|---|---|---|---|
| | Elemental Analysis | | | | | | BET | Micro-Pore |
| Cata- | (wt. %) | | | (SiₓAl_{y}P_{z})O₂ | | | SA, | Area, |
| lyst | % Si | % Al | % P | x | y | z | m₂/g | m²/g |
| A | 6.77 | 23.86 | 15.63 | 0.15 | 0.54 | 0.31 | 546 | 525 |
| B | 3.22 | 23.46 | 15.21 | 0.08 | 0.59 | 0.33 | — | — |
| C | 6.44 | 23.35 | 17.80 | 0.14 | 0.52 | 0.34 | 454 | 419 |
| D | 4.01 | 27.31 | 15.33 | 0.09 | 0.61 | 0.30 | 525 | 448 |

X-ray powder diffraction patterns of the air-calcined SAPO-34 powders were recorded on a Philips PANanalytical X'Pert XRD System using CuKα radiation. The peak positions, d-spacing and the peak relative intensities of the catalyst samples are given in Tables 4 to 7.

TABLE 4
XRD peaks and intensities for Catalyst A

| 2Theta (degree) | d-spacing | Intensity* |
|---|---|---|
| 9.53 | 9.28 | 100.00 |
| 12.92 | 6.85 | 26.90 |
| 16.06 | 5.52 | 10.70 |
| 17.80 | 4.98 | 13.74 |
| 20.65 | 4.30 | 46.98 |
| 23.10 | 3.85 | 6.67 |
| 24.98 | 3.56 | 19.66 |
| 25.97 | 3.43 | 12.15 |
| 28.21 | 3.16 | 4.57 |
| 30.67 | 2.92 | 31.30 |
| 31.13 | 2.87 | 17.59 |
| 34.56 | 2.60 | 5.82 |
| 36.08 | 2.49 | 4.50 |

*Intensities shown are scaled in arbitrary units so that most intense peak is 100.

TABLE 5
XRD peaks and intensities for Catalyst B

| 2Theta (degree) | d-spacing | Intensity* |
|---|---|---|
| 9.61 | 9.21 | 100.00 |
| 13.05 | 6.78 | 30.70 |
| 16.19 | 5.47 | 19.87 |
| 17.89 | 4.96 | 17.67 |
| 19.24 | 4.61 | 5.58 |
| 20.85 | 4.26 | 48.50 |
| 23.28 | 3.82 | 10.01 |
| 25.12 | 3.54 | 21.03 |
| 26.24 | 3.40 | 19.04 |
| 28.37 | 3.15 | 8.65 |
| 29.94 | 2.98 | 5.70 |
| 31.01 | 2.88 | 44.11 |
| 31.32 | 2.86 | 25.28 |
| 34.91 | 2.57 | 5.11 |

*Intensities shown are scaled in arbitrary units so that most intense peak is 100.

TABLE 6
XRD peaks and intensities for Catalyst C

| 2Theta (degree) | d-spacing | Intensity* |
|---|---|---|
| 9.61 | 9.21 | 100.00 |
| 13.04 | 6.78 | 29.87 |
| 16.22 | 5.47 | 21.63 |
| 17.92 | 4.95 | 11.61 |
| 18.75 | 4.73 | 8.63 |
| 19.21 | 4.62 | 7.54 |
| 20.83 | 4.26 | 56.60 |
| 23.30 | 3.82 | 14.65 |
| 25.18 | 3.54 | 17.58 |
| 26.19 | 3.40 | 22.82 |
| 28.01 | 3.19 | 6.38 |
| 28.44 | 3.14 | 6.80 |
| 29.88 | 2.99 | 6.76 |
| 30.95 | 2.89 | 48.04 |
| 31.35 | 2.85 | 33.97 |
| 34.93 | 2.57 | 6.16 |
| 49.41 | 1.84 | 6.18 |

*Intensities shown are scaled in arbitrary units so that most intense peak is 100.

TABLE 7
XRD peaks and intensities for Catalyst D

| 2Theta (degree) | d-spacing | Intensity* |
|---|---|---|
| 9.51 | 9.30 | 100.00 |
| 12.90 | 6.86 | 27.55 |
| 16.06 | 5.52 | 15.83 |
| 17.83 | 4.97 | 11.54 |
| 20.65 | 4.30 | 48.13 |
| 23.13 | 3.85 | 7.43 |
| 25.03 | 3.56 | 14.60 |
| 26.00 | 3.43 | 13.20 |
| 30.71 | 2.91 | 29.03 |
| 31.16 | 2.87 | 17.45 |
| 34.61 | 2.59 | 4.70 |

*Intensities shown are scaled in arbitrary units so that most intense peak is 100.

Magic angle spinning (MAS) solid state ²⁹Si NMR studies were performed on the calcined SAPO-34 catalysts. Measurements used 270 MHz spectrometer (²⁹Si at 53.762 MHz), room temperature, 7 mm rotors, spinning speed about 4 kHz, 45 degree pulse, 60 second delay and 200-600 scans signal averaging. All spectra were referenced to tetramethylsilane (TMS) at a 0.0 ppm on the chemical shift scale.

MAS solid state ²⁷Al and ³¹P NMR data were both collected on a 363 MHz instrument with ²⁷Al at 94.669 MHz and ³¹P at 147.085 MHz. All ²⁷Al data were collected with a 15 degree pulse length and a recycle delay of 300 ms. 7000-10000 scans were collected for each sample. The rotor size was 5 mm and spinning speed was 10 kHz. Chemical shifts were referenced to external 1M Al(NO₃)₃ at 0.00 ppm. Whereas ³¹P data were collected with a 45 degree pulse length and a recycle delay of 10 s. Approximately 300 scans were collected for each sample. The rotor size was 7 mm and the spinning speed was 7 kHz. The chemical shifts were referenced to external 85% H₃PO₄ at 0.00 ppm.

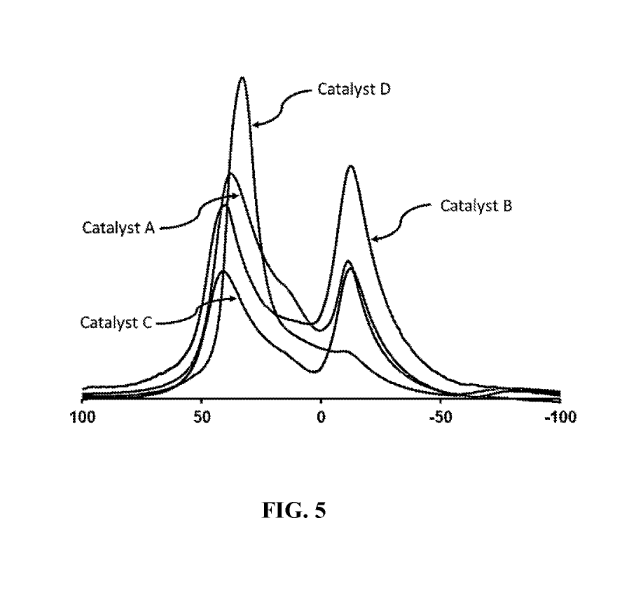

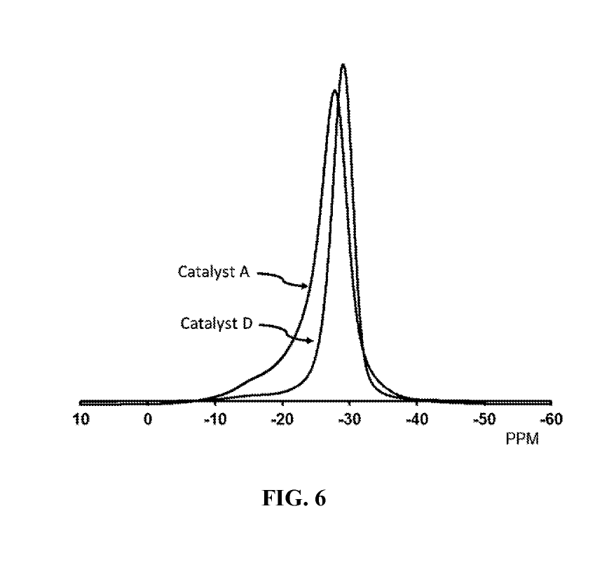

FIG. 3 shows MAS ²⁹Si NMR spectra of comparative catalysts A and B. Both comparative catalysts show the Si(4Al) peak at about −90 ppm. FIG. 4 shows MAS ²⁹Si NMR spectra of example catalysts C and D. As shown in FIG. 4, the catalyst C of the invention shows Si(4A), Si(3Al), Si(2Al), Si(1Al) and Si(0Al) peaks at about −89, −94, −99, −106 (shoulder), and −110 ppm, respectively. The catalyst D of the invention shows only one Si(3Al) peak at −94 ppm. FIG. 5 shows MAS ²⁷Al NMR spectra of catalysts A, B, C and D, whereas FIG. 6 shows MAS ³¹P NMR spectra of catalysts A and D.

### Chloromethane Conversion Reactions of Catalysts A-D

Each of the Catalysts A through D was tested for chloromethane conversion by using a fixed-bed tubular reactor at about 350° C. for a period of about 20 h or longer. For catalytic test the powder catalyst was pressed and then crushed and sized between 20 and 40 mesh screens. In each test a fresh load of sized (20-40 mesh) catalyst (3.0 g) was loaded in a stainless steel tubular (½-inch outer diameter) reactor. The catalyst was dried at 200° C. under N₂ flow (100 cm³/min) for an hour and then raised to 300° C. at which time N₂ was replaced by methyl chloride feed (90 cm³/min) containing 20 mol % CH₃Cl in N₂ was introduced to the reactor. The weight hourly space velocity (WHSV) of CH₃Cl was about 0.8 h⁻¹ to 1.0 h⁻¹ and reactor inlet pressure was about 1 to 3 psig. The reaction temperature was ramped to 350° C. after about 2-3 h of initial reaction period. Reaction conditions are summarized in Table 8. The pre- and post-run feeds were analyzed and the average was taken into calculations for catalyst performance.

TABLE 8
Reaction conditions

| | | | | | | Reactor |
|---|---|---|---|---|---|---|
| | | Cata- | Feed | | | Inlet |
| | Cata- | lyst | Rate¹ | WHSV | Temp | Pressure |
| Example | lyst | (g) | (cm3/min) | (h⁻¹) | (° C.) | (psig) |
| 1 | A | 3.01 | 90 | 0.9 | 349 | 2.5 |
| (Comparative | | | | | | |
| Example) | | | | | | |
| 2 | B | 3.01 | 90 | 0.83 | 350 | 1.8 |
| (Comparative | | | | | | |
| Example) | | | | | | |
| 3 | C | 3.01 | 90 | 0.81 | 349 | 1.3 |
| (Example of the | | | | | | |
| invention) | | | | | | |
| 4 | D | 3.0 | 90 | 0.83 | 350 | 1.6 |
| (Example of the | | | | | | |
| invention) | | | | | | |

¹Total feed rate (feed contains 20 mol % CH₃Cl in N₂).

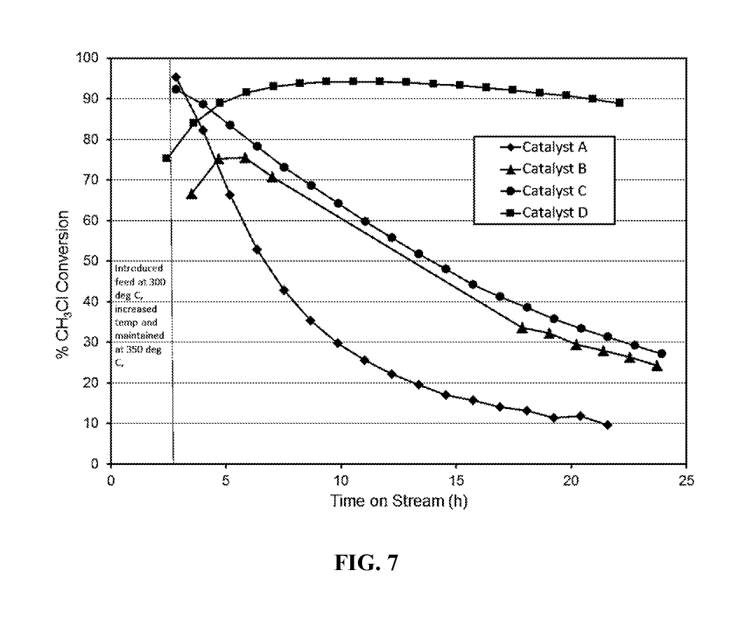

FIG. 7 shows conversion of CH₃Cl over catalysts A-D. The conversion of chloromethane and selectivity of olefins were calculated using Equations (IV)-(VII) shown earlier. Table 9 below provides the CH₃Cl conversion and selectivity to ethylene, propylene and butylenes at 20 h run time for the comparative catalysts A and B, and example catalysts C and D of the present invention. The conversion data provides information about catalyst performance stability—higher the conversion better the catalyst stability for the reaction. Comparative catalysts A and B show about 12 and 29% CH₃Cl conversions at 20 h. Whereas the example catalysts C and D show about 35% and 91% conversions at 20 hour. The selectivity to C₂-C₄ olefins were 88-95% for the comparative catalysts A and B; and about 95% C₂-C₄ olefin selectivity for both the example catalysts C and D.

TABLE 9
Conversion and selectivity data

| | % CH₃Cl | Selectivity (% C-mol) at 20 h | | | |
|---|---|---|---|---|---|
| Catalyst | Conversion at 20 h | C₂H₄ | C₃H₆ | C₄H₈ | C₂-C_{4 Olefins} |
| A | 11.8 | 50.5 | 32.1 | 5.9 | 88.5 |
| B | 29.4 | 47.1 | 40.8 | 6.9 | 94.8 |
| C | 34.5 | 50.2 | 40.5 | 5.0 | 95.7 |
| D | 90.7 | 40.4 | 42.9 | 11.1 | 94.4 |

From the examples shown above, Catalyst D of the present invention, which has the desired SAPO-34 structure containing silicon coordinated with three or less aluminum atom via oxygen atoms as shown by ²⁹Si magic angle spinning (MAS) nuclear magnetic resonance (NMR) spectroscopy peak(s), is the most stable catalyst having a conversion rate of over 90% after 20 hours on stream. Further, the selectivity of C₂-C₄ olefins is over 90%. Catalysts having this level of stability and selectivity for C₂-C₄ olefins offer significant advantages ranging from lower production costs to an increase in C₂-C₄ production over the same time period when compared to currently available SAPO-34 catalysts.

1. A catalyst capable of producing an olefin from an alkyl halide, the catalyst comprising a silicoaluminophosphate (SAPO) having a chabazite zeolite structure with the following chemical composition:
(SiₓAl_{y}P_{z})O₂
where x, y, and z represent the mole fractions of silicon, aluminum, and phosphorus, respectively, present as tetrahedral oxides, x is 0.01 to 0.30 and the sum of x+y+z is 1, and
where the catalyst comprises silicon tetrahedral oxides that are connected with three or less aluminum tetrahedral oxide as shown by ²⁹Si magic angle spinning (MAS) nuclear magnetic resonance (NMR) spectroscopy peak(s) with peak(s) maxima between −93 ppm and −115 ppm, and
wherein the catalyst includes 25% or less of silicon tetrahedral oxide shared by four aluminum tetrahedral oxides.

2. The catalyst of claim 1, where a majority of the silicon tetrahedral oxides in the crystal lattice are connected with three or less aluminum tetrahedral oxides.

3. (canceled)

4. The catalyst of claim 1, where each silicon tetrahedral oxide is connected with three tetrahedral oxides.

5. The catalyst of claim 1, wherein the peak maxima is between −93 ppm and −97 ppm.

6. The catalyst of claim 1, wherein y is 0.40 to 0.60 and z is 0.25 to 0.49.

7. The catalyst of claim 1, wherein the catalyst is capable of converting 30 to 95% alkyl halide after 20 hours of use at a temperature of 325 to 375° C., a WHSV of between 0.5 and 6.0 h⁻¹, and a pressure of 1 to 4 psig.

8. (canceled)

9. The catalyst of claim 7, having a selectivity of ethylene, propylene, and butylene of at least 90% after 20 hours of use.

10. (canceled)

11. The catalyst of claim 1, wherein the catalyst has been calcined at a temperature of 400 to 600° C.

12. The catalyst of claim 1, characterized by a powder x-ray diffraction pattern as substantially depicted in Table 6, or Table 7.

13. (canceled)

14. A method for converting an alkyl halide to an olefin, the method comprising contacting any one of catalysts of claim 1 with a feed comprising an alkyl halide under reaction conditions sufficient to produce an olefin hydrocarbon product.

15. The method of claim 14, where each silicon tetrahedral oxide is coordinated with three aluminum tetrahedral oxides as shown by ²⁹Si MAS NMR spectroscopy peak maxima between −93 ppm and −97 ppm or −94 ppm to −95 ppm.

16. (canceled)

17. (canceled)

18. The method of claim 14, wherein the catalyst converts 30 to 95% alkyl halide after 20 hours of use at a temperature of 325 to 375° C., a WHSV of between 0.5 and 6.0 h⁻¹, and a pressure of 1 to 4 psig.

19. The method of claim 18, wherein the catalyst converts 90 to 95% alkyl halide after 20 hours of use at a temperature of 325 to 375° C., a WHSV of between 0.5 and 6.0 h⁻¹, and a pressure of 1 to 4 psig.

20. The method of claim 18, wherein the catalyst has a selectivity of ethylene, propylene, and butylene of at least 90% after 20 hours of use.

21. (canceled)

22. The method of claim 14, wherein the catalyst has been previously calcined at a temperature of 400 to 600° C.

23. The method of claim 14, wherein the alkyl halide is a methyl halide.

24. The method of claim 23, wherein the feed comprises about 10 mole % or more of the methyl halide.

25. The method of claim 23, wherein the methyl halide is methyl chloride, methyl bromide, methyl fluoride, or methyl iodide, or any combination thereof.

26. The method of claim 23, wherein the catalyst has not been subjected to a halide treatment.

27. (canceled)

28. (canceled)

29. The method of claim 14, further comprising regenerating the used catalyst after 20, 25, or 30 hours of use.

30-33. (canceled)
